(19) United States
(12) Patent Application Publication
(10) Pub. No.: US 2026/0295048 A1
(43) Pub. Date: Oct. 1, 2026

(54) MAGNETIC HYDROGEL MATERIAL WITH BOTH BACTERICIDAL PROPERTY AND THERMORESPONSIVE CONTRACTILITY, PREPARATION METHOD AND APPLICATION THEREOF

(71) Applicant: The First Affiliated Hospital of Xi'an Jiaotong University, Xi'an (CN)

(72) Inventors: Huicong Du, Xi'an (CN); Maoguo Shu, Xi'an (CN)

(21) Appl. No.: 19/455,852

(22) Filed: Jan. 22, 2026

(30) Foreign Application Priority Data
Mar. 25, 2025 (CN) 2025103575954

(51) Int. Cl. A61K 41/00; A61K 9/06; A61K 47/14; A61K 47/18; A61K 47/34; A61K 47/46; A61P 17/02

(52) U.S. Cl. CPC A61K 41/0052; A61K 9/06; A61K 47/14; A61K 47/18; A61K 47/34; A61K 47/46; A61P 17/02

(57) ABSTRACT

A magnetic hydrogel material with both bactericidal property and thermoresponsive contractility, a preparation method and an application thereof are provided. The magnetic hydrogel material adopts a poly(N-isopropylacrylamide) (PNIPAm) copolymerized polyacrylamide (PAm) double-network hydrogel as a matrix. Nano clay is grafted on the matrix to form a copolymer double-network hydrogel with thermoresponsive contractility, and a magnetic substance and tannic acid are doped in the copolymer double-network hydrogel to obtain the magnetic hydrogel material. The PNIPAm copolymerized PAm double-network hydrogel is formed through a radical polymerization reaction of N-isopropylacrylamide and acrylamide. The magnetic hydrogel material has both bactericidal property and mechanical loading property. The magnetic hydrogel material can effectively kill Staphylococcus aureus and Escherichia coli, and promote the healing of the infected diabetic wounds through bactericidal property and sustained mechanical loading on wounds.

### CROSS-REFERENCE TO RELATED APPLICATION

This application claims priority to Chinese patent application No. CN202510357595.4, filed to China National Intellectual Property Administration (CNIPA) on Mar. 25, 2025, which is herein incorporated by reference in its entirety.

### TECHNICAL FIELD

The disclosure relates to the field of hydrogel technologies, and in particular to a magnetic hydrogel material with both bactericidal property and thermoresponsive contractility, a preparation method and an application thereof.

### BACKGROUND

As a global chronic metabolic disorder, the number of diabetes patients is expected to reach 643 million by 2030 and 783 million by 2045. Diabetic dermatopathy is one of the most common complications of diabetes, with an estimated prevalence of 51.1% to 97.0%. Even minor skin injuries can lead to infected diabetic wounds in patients with diabetes. These wounds are prone to infection and formation of biofilms, which can hinder the healing process. Due to the complex wound microenvironment, treatment of the infected diabetic wounds remains a major clinical challenge.

Although some progress has been made in treatment of chronic diabetic wounds, challenges such as low bioavailability, complex disease microenvironment and insufficient drug delivery efficiency still limit therapeutic effect. Therefore, combination therapies capable of integrating multiple functions, such as coordinated anti-infection effect, good biocompatibility and promotion of wound healing, have shown a prospect of accelerating the healing of the infected diabetic wounds. Given an increasing prevalence of multidrug-resistant pathogens and recalcitrant pathogenic biofilms, conventional antibiotics have been clinically proven to have limited therapeutic efficacy.

In addition to magnetic field and magnetic attraction property, magnetic materials also exhibit magnetocaloric effect, which refer to phenomena where temperatures of magnets or paramagnetic materials change with magnetic field strength. Typically, under adiabatic conditions, magnetization causes an increase in temperature of the magnetic materials, while demagnetization causes a decrease in temperature of the magnetic materials. By utilizing the magnetocaloric effect, the temperatures of the magnetic materials can be tuned to desired levels by adjusting the magnetic field strength. Researches have shown that heating to 50 Celsius degrees (° C.) for 20 minutes can effectively kill bacteria. In addition, thermoresponsive hydrogels are widely used in biomedical fields based on their temperature responsive property. The healing of the wounds is closely related to skin contraction, and surgical suturing and skin stretching techniques also promote the healing of the wounds by applying external force to shrink the wounds.

Therefore, there is an urgent need for a hydrogel material with both bactericidal and thermoresponsive contraction to play a combined effect of anti-infection and mechanical loading to promote the healing of the wounds through the thermoresponsive contraction of the material.

### SUMMARY

The disclosure aims to provide a magnetic hydrogel (MHd) material with both bactericidal property and thermoresponsive contractility, a preparation method and an application thereof.

To achieve the above objectives, the disclosure employs the following technical solutions.

In a first aspect, the disclosure provides a magnetic hydrogel material with both bactericidal property and thermoresponsive contractility. A poly(N-isopropylacrylamide) (PNIPAm) copolymerized polyacrylamide (PAm) double-network hydrogel is used as a matrix. Nano clay is grafted on the matrix to form a copolymer double-network hydrogel with thermoresponsive contractility, and a magnetic substance and tannic acid (TA) are doped in the copolymer double-network hydrogel with thermoresponsive contractility to obtain the magnetic hydrogel material.

The PNIPAm copolymerized PAm double-network hydrogel is formed through a radical polymerization reaction of N-isopropylacrylamide (NIPAm) and acrylamide (Am).

The magnetic hydrogel material of the disclosure improves the lowest critical solution temperature (LCST) of a PNIPAm-based hydrogel by doping the nano clay and using the PNIPAm copolymerized PAm double-network hydrogel. The magnetic hydrogel material has magnetocaloric effect by introducing triiron tetroxide (Fe₃O₄, also referred to as magnetite). The TA is introduced to enhance a bactericidal effect of the magnetic hydrogel material. Through synergistic action of components of the magnetic hydrogel material, the magnetic hydrogel material can simultaneously provide stable mechanical loading at a higher bactericidal temperature, and thereby enabling a combined strategy of coupled hyperthermia-based antibacterial therapy and mechanical healing.

In an embodiment, the magnetic substance is Fe₃O₄.

In a second aspect, the disclosure provides a preparation method of the magnetic hydrogel material, including the following steps:
dissolving the NIPAm, a catalyst, the Am, a crosslinking agent, the nano clay, the magnetic substance, and the TA in water to prepare a homogeneous precursor solution; and
adding an initiator and an accelerator to the homogeneous precursor solution in a protective gas atmosphere, followed by mixing evenly and polymerizing for 12 hours (h) to 16 h at 4° C. to 7° C. to obtain the magnetic hydrogel material.

In an embodiment, mass concentrations of the NIPAm, the catalyst, the Am, the crosslinking agent, the nano clay, the magnetic substance, and the TA in the homogeneous precursor solution are 8%-12%, 0.2%-0.5%, 1%-3%, 0.1%-0.5%, 3%, 15%, and 1%, respectively.

In an embodiment, the catalyst is sodium pyrophosphate (TSSP), the crosslinking agent is N,N′-methylenebisacrylamide (MBAA), the initiator is tetramethylethylenediamine (TMEDA), and the accelerator is an ammonium persulfate solution (APS) with a mass concentration in a range 28% to 32%.

In an embodiment, a volume ratio of the water to the initiator to the accelerator is 1000:(1-2.5):(4-6).

In a third aspect, the disclosure provides an application of the magnetic hydrogel material in preparation of a bactericidal product.

In an embodiment, the bactericidal property includes killing Staphylococcus aureus and killing Escherichia coli.

In a fourth aspect, the disclosure provides an application of the magnetic hydrogel material in preparation of a dressing for promoting healing of infected diabetic wounds.

In a fifth aspect, the disclosure provides a dressing for promoting the healing of infected diabetic wounds, including the magnetic hydrogel material.

Compared with the related art, the disclosure has the following beneficial effects.

The disclosure employs the magnetocaloric effect to prepare the magnetic hydrogel material with both bactericidal property and mechanical loading property. Experimental results show that the magnetic hydrogel material can effectively kill Staphylococcus aureus and Escherichia coli, and effectively promote the healing of the infected diabetic wounds through bactericidal property and sustained mechanical loading on the wounds.

### BRIEF DESCRIPTION OF DRAWINGS

FIG. 1 illustrates a rheological characterization diagram of nano clay with mass concentrations of 1%, 2%, and 3% in a precursor solution according to embodiment 1 of the disclosure.

FIGS. 2A-2B illustrate stress-strain curves and rheological characterization of a material system with sequential addition of Fe₃O₄ and TA according to the embodiment 1 of the disclosure. Specifically, FIG. 2A illustrates a stress-strain curve diagram of the material system with sequential addition of Fe₃O₄ and TA; and FIG. 2B illustrates a rheological characterization diagram of the material system with sequential addition of Fe₃O₄ and TA.

FIGS. 3A-3C illustrate observation diagrams of a network structure of an MHd material using a scanning electron microscope (SEM) according to embodiment 2 of the disclosure. Specifically, FIG. 3A illustrates a SEM diagram at a magnification of 100 times, FIG. 3B illustrates a SEM diagram at a magnification of 200 times, and FIG. 3C illustrates a SEM diagram at a magnification of 4000 times.

FIGS. 4A-4B illustrate tensile test diagrams of the MHd material according to embodiment 3 of the disclosure. Specifically, FIG. 4A illustrates an actual tensile test diagram, and FIG. 4B illustrates a stress-strain curve diagram.

FIG. 5 illustrates a real-time infrared thermal imaging detection diagram of magnetocaloric effect generated by the MHd material under a high-frequency alternating magnetic field according to embodiment 4 of the disclosure.

FIG. 6 illustrates an intuitive display diagram of area changes of the MHd material after thermoresponsive contraction according to embodiment 5 of the disclosure.

FIG. 7 illustrates a percentage change diagram in area of the MHd material after thermoresponsive contraction according to the embodiment 5 of the disclosure.

FIG. 8 illustrates a bactericidal effect diagram of the MHd material on Staphylococcus aureus and Escherichia coli according to embodiment 6 of the disclosure.

FIG. 9 illustrates a biocompatibility testing diagram of the MHd material according to embodiment 7 of the disclosure.

FIGS. 10A-10B illustrate wound healing performance of the MHd material in a diabetic mouse model with Staphylococcus aureus-infected wounds according to embodiment 8 of the disclosure. Specifically, FIG. 10A illustrates a schematic diagram of an animal experiment, and FIG. 10B illustrates wound healing status of the wounds at different time points.

### DETAILED DESCRIPTION OF EMBODIMENTS

The disclosure will be described in detail below in conjunction with the accompanying drawings and specific embodiments, but the embodiments should not be construed as a limitation of the disclosure. Unless otherwise specified, the technical means used in the following embodiments are conventional means well-known to those skilled in the art. The materials, reagents, etc. used in the following embodiments can be obtained from commercial sources unless otherwise specified.

### Embodiment 1: Preparation of an MHd Material with Both Bactericidal Property and Thermoresponsive Contractility

A PNIPAm-PAm/Fe₃O₄/Clay/TA magnetic hydrogel is prepared through a one-pot method of radical polymerization. Specifically, the one-pot method includes the following steps: NIPAm, TSSP, Am, MBAA, Clay (i.e., nano clay), Fe₃O₄ and TA are dissolved in 20 milliliters (mL) deionized water sequentially in that order to prepare a homogeneous precursor solution. A concentration of each component in the precursor solution is shown in Table 1. The precursor solution is swept with nitrogen in an ice water bath for 30 minutes to obtain a swept precursor solution. TMEDA (30 microliters abbreviated as μL) and APS (100 μL) with a mass concentration of 30% are added to the swept precursor solution, followed by mixing well to obtain a hydrogel precursor solution. The hydrogel precursor solution is moved into a mold, followed by polymerizing at 4° C. for 12 hours to obtain a prepared sample (i.e., the magnetic hydrogel material).

TABLE 1
Composition of the magnetic hydrogel material

| Component | NIPAm | TSSP | Am | MBAA | Clay | Fe₃O₄ | TA |
|---|---|---|---|---|---|---|---|
| Concentration (w/v) | 8-12% | 0.2-0.5% | 1-3% | 0.1-0.5% | 3% | 15% | 1% |

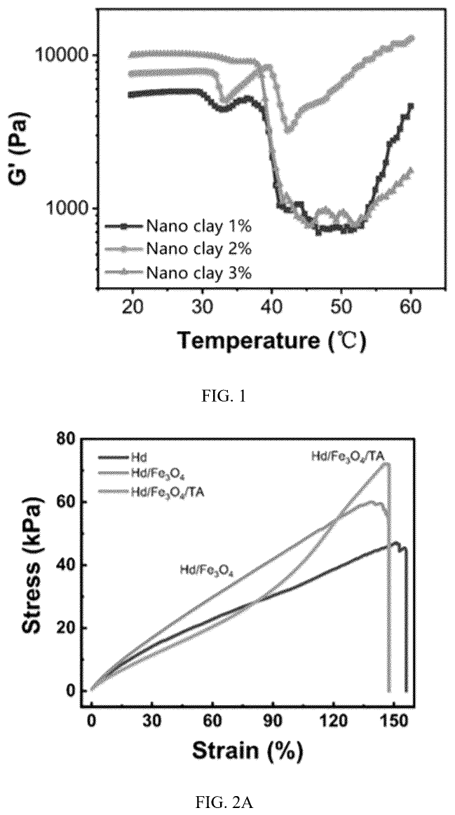

For a mechanical active dressing based on thermoresponsive hydrogel network, an intervention temperature excessively higher than LCST will lead to rapid contraction of the dressing itself and separation from adhesive skin tissue, so that mechanical stimulation generated by the contraction of the dressing cannot be transmitted to a wound. The LCST of a PNIPAM-based hydrogel falls within a range of approximately 30° C. to 32° C. An excessively high intervention temperature will cause rapid dehydration of the PNIPAM-based hydrogel, thereby making the PNIPAM-based hydrogel difficult to adhere to skin tissue. However, diabetic wounds are often accompanied by infection. In order to combine thermoresponsive contraction with hyperthermia-based antibacterial therapy, it is often necessary to appropriately adjust the LCST of a thermoresponsive hydrogel, so as to ensure that the mechanical force generated by material contraction can still be stably transmitted to wound tissue through adhesion under a higher intervention temperature (i.e., a temperature sufficient to kill bacteria). In this embodiment, the LCST of the PNIPAM-based hydrogel is improved by doping the nano clay and using a PNIPAm copolymerized PAm double-network hydrogel, so that the mechanically active dressing can simultaneously provide stable mechanical stimulation at a higher bactericidal temperature to thereby realize a combined strategy of coupled hyperthermia-based antibacterial therapy and mechanical healing. This embodiment first enhances the LCST by doping the nano clay, with the mass concentration of the nano clay in the precursor solution as a single factor, and the mass concentrations of other components fixed at NIPAm (10.5%), TSSP (0.35%), Am (2%), MBAA (0.25%), Fe₃O₄ (15%), TA (1%) to verify an enhancing effect of doped nano clay on the LCST of the MHd material. As a doping ratio of the nano clay increases from 1%, 2% to 3%, the LSCT increases from 33° C. (1%) to 38° C. (as shown in FIG. 1). Moreover, due to its special lamellar structure, the nano clay provides more crosslinking sites for the hydrogel network and reduces a use of a cross-linking agent (MBAA). With the increase of content of the nano clay, storage modulus (G′) of the hydrogel is also improved.

In order to give the material magnetocaloric effect, Fe₃O₄ is introduced into the system. To enhance a bactericidal effect of the material, TA is added. Mechanical property of the material is evaluated and Young's modulus is calculated through tensile-strain experiments.

Experimental groups are set as follows: (1) a hydrogel (Hd) group: the components and the mass concentrations in the precursor solution are NIPAm 10.5%, TSSP 0.35%, Am 2%, MBAA 0.25%, and Clay 3%; (2) a Hd/Fe₃O₄ group: the components and the mass concentrations in the precursor solution are NIPAm 10.5%, TSSP 0.35%, Am 2%, MBAA 0.25%, Clay 3%, and Fe₃O₄ 15%; (3) a Hd/Fe₃O₄/TA group: the components and the mass concentrations in the precursor solution are NIPAm 10.5%, TSSP 0.35%, Am 2%, MBAA 0.25%, Clay 3%, Fe₃O₄ 15%, and TA of 1%. The preparation method is as described above.

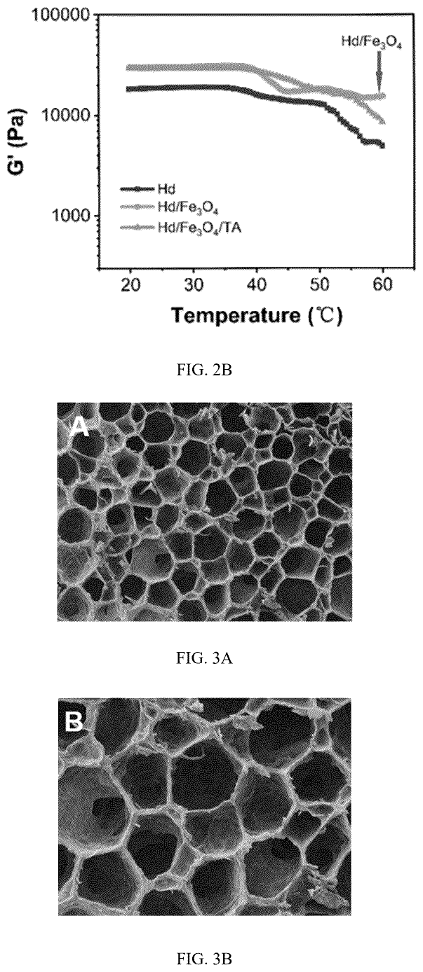

Experimental results are shown in FIGS. 2A-2B. With the introduction of Fe₃O₄ and TA, the mechanical property of the hydrogel are significantly improved. Rheological characterization shows that the introduction of Fe₃O₄ and TA does not significantly change the LCST of the hydrogel.

Subsequently, the MHd prepared from the Hd/Fe₃O₄/TA group is used as samples for structural characterization and functional identification.

### Embodiment 2: Morphology Structural Characterization of the MHd Material

The samples are freeze-dried to evaluate microstructure of the hydrogel. The JSM-IT700HR scanning electron microscope produced by Japan Electron Optics Laboratory Co., Ltd. (JEOL) is used to collect images of the samples. Each sample is first observed in its entirety at low magnification, and then an area to be observed is selected to collect images to observe the specific structure.

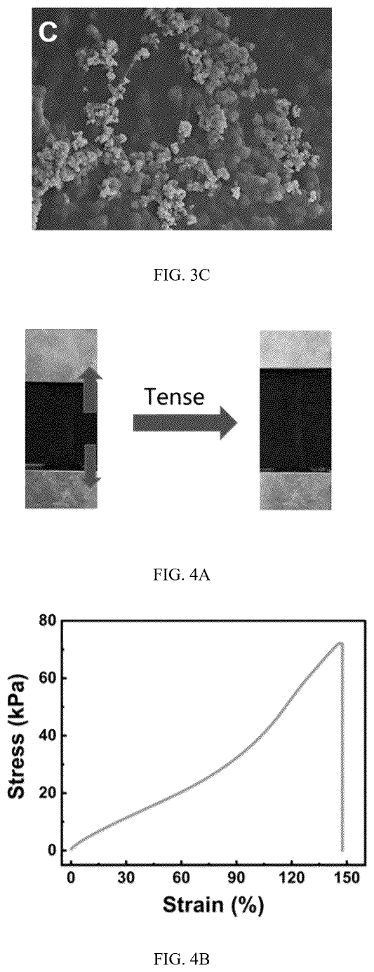

By evaluating its microstructure through scanning electron microscopy (as shown in FIGS. 3A-3C), it can be seen that the prepared MHd material has a network structure with uniform pore size, and the Fe₃O₄ and the nano clay are homogeneously dispersed in the network, ensuring that the material can have an isotropic mechanical property.

### Embodiment 3: Mechanical Property Testing of the MHd Material

Two ends of the prepared MHd material with a dumbbell shape (tensile sample) are fixed on a holder of an electronic universal testing machine (Shenzhen Sansizongheng Technology, UTM6202). An initial position of the holder is adjusted to make the prepared sample fully extended and free of tension (as shown in FIG. 4A). The universal testing machine is zeroed and calibrated, and then the holder is moved at a constant speed of 5 millimeters per minute (mm/min) to stretch the sample until the sample breaks. A maximum stress is recorded at a time when the sample is fractured, and a stress-strain curve (as shown in FIG. 4B) is drawn based on tensile stress data and tensile length data of corresponding sample. The Young's modulus is determined through the stress-strain curve.

The Young's modulus of the magnetic hydrogel material is 48.84 kilopascals (kPa), which indicates that the material has high mechanical property and can meet the subsequent mechanical loading.

### Embodiment 4: Magnetocaloric Property Testing of the MHd Material

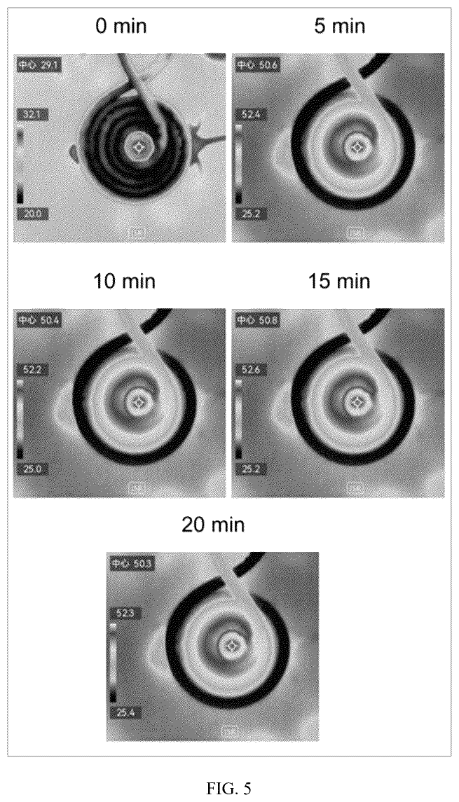

A disc-shaped coil of a high-frequency induction heating machine (e.g. a model of HNG-15KW, Huaneng Power Technology, Shenzhen) is used to provide high-frequency alternating magnetic field (AMF) for magnetic induction heating of the MHd material. An infrared thermal imager (e.g. a model of K₂O, Hikmicro) is used to measure and record real-time temperatures of the MHd material. Testing results show that the MHd material can be heated to 50° C. and kept stable for 20 minutes through the high-frequency AMF (as shown in FIG. 5).

### Embodiment 5: Thermoresponsive Contractility Testing of the MHd Material

The disc-shaped coil of the high-frequency induction heating machine (e.g. the model of HNG-15KW, Huaneng Power Technology, Shenzhen) is used to provide a high-frequency AMF for magnetic induction heating of the MHd material. The MHd material is kept at a temperature of 50° C., an area change of the material is recorded every 5 minutes with a camera, and contraction percentage of the material is calculated.

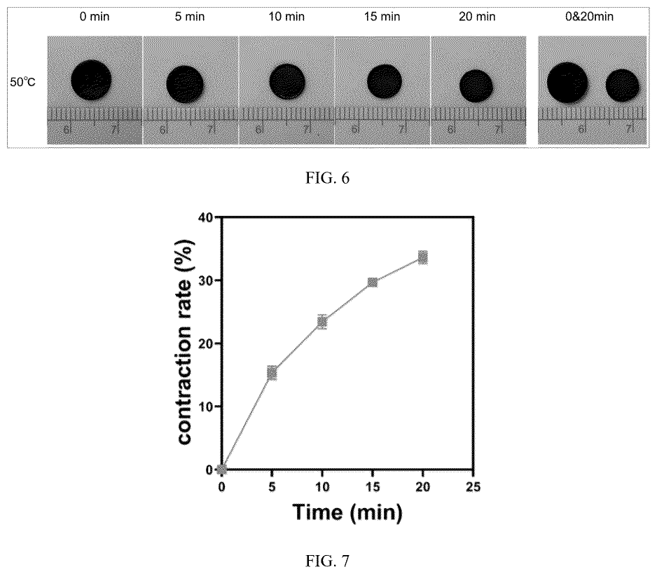

Calculation results for area contraction percentage of the MHd material show that the area contraction percentage gradually increases over time when heated to 50° C. (as shown in FIG. 6), and reaches 33% at 20 minutes (as shown in FIG. 7). The testing indicates that the material has good thermoresponsive contractility.

### Embodiment 6: Bactericidal Property Testing of the MHd Material

Firstly, 100 μL sterile MHd material is prepared and placed in a 12-well plate. Then, 20 μL Staphylococcus aureus (S. aureus) suspension with a concentration of 1×10⁸ colony forming units per milliliter (CFU/mL) and 20 μL Escherichia coli (E. coli) suspension with a concentration of 1×10⁸ CFU/mL are respectively inoculated onto a surface of the MHd material. At the same time, a negative control group is set up, that is, the same amount (20 μL) of bacterial suspension is inoculated under a non-hydrogel condition. An MHd (50° C.) group and the negative control group are both placed in a high-frequency AMF for processing, and the temperature of the MHd material is raised to 50° C. and maintained for 20 minutes through magnetocaloric effect. While an MHd (−) group is not placed in a high-frequency AMF for processing. After interventions are completed, remaining bacterial solutions from each group are collected and sufficiently resuspended, and a plate coating method is used for colony counting to evaluate bacterial survival rates.

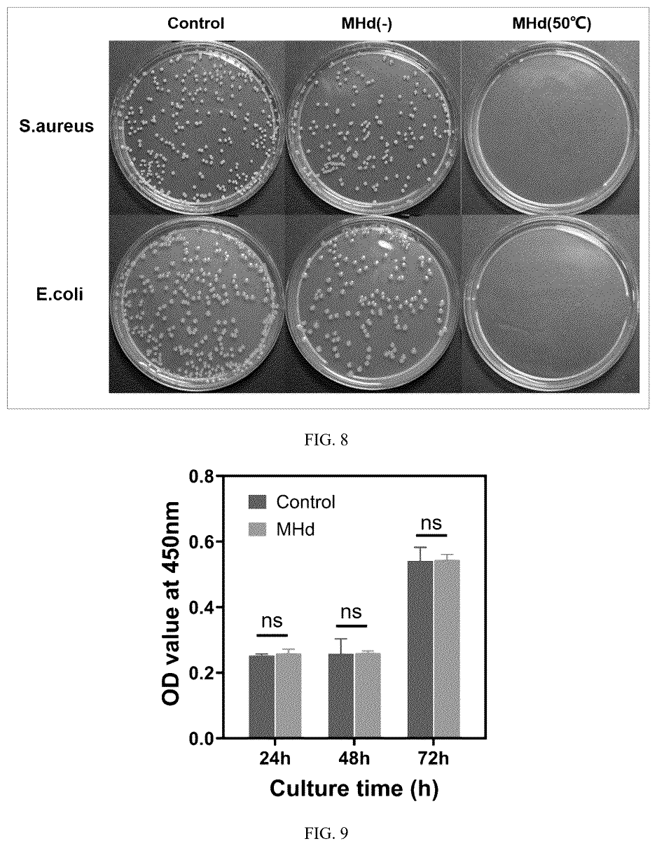

Bactericidal property of the MHd material is measured in vitro, and results show that, in the negative control group (Control), colonies of S. aureus and E. coli grow extensively across the plate. The MHd (−) group only partially inhibits bacterial growth, whereas the MHd (50° C.) group exhibits a high bactericidal efficiency of 99% against S. aureus and E. coli, with almost no surviving bacterial colonies observed on the plate (as shown in FIG. 8). The testing indicates that by applying a high-frequency AMF to raise the temperature of the MHd material to 50° C., a highly synergistic bactericidal effect is achieved through a use of magnetocaloric effect and an explosive release of TA at 50° C., while the bactericidal effect solely relying on TA is very limited.

### Embodiment 7: Biocompatibility Testing of the MHd Material

An experiment is conducted by culturing NIH-3T3 (i.e., mouse embryonic fibroblasts) in vitro, and the experiment is divided into a control group and an MHd material group. After further sterilization using ultraviolet (UV) irradiation, the MHd material prepared under a sterile condition is soaked in complete Dulbecco's modified Eagle medium (DMEM) medium for more than one week to obtain complete DMEM medium containing MHd material leachate. NIH-3T3 cells are seeded in a 96-well plate and divided into two groups: the control group is added with normal complete DMEM medium, and the MHd material group is added with complete DMEM medium containing MHd material leachate. The NIH-3T3 cells are cultured for 24 hours, 48 hours, and 72 hours respectively. Cell Counting Kit-8 (CCK-8) reagents are added into the corresponding wells. The NIH-3T3 cells are incubated at 37° C. for 2 hours, and cell viability is detected through measuring optical density at 450 nanometers (OD450).

Due to a subsequent need to attach the MHd material to the wounds, the biocompatibility of the material is evaluated. CCK-8 detection results show that the optical density (OD) value of the control group and the MHd material group are almost the same at 24 hours, 48 hours, and 72 hours of culture, without statistical difference, indicating that the MHd material has no effect on cell activity. With increase of cultivation time, the OD value of cells at 72 hours significantly increases, proving that the MHd material does not hinder cell proliferation (as shown in FIG. 9). The above results demonstrate that the MHd material have good biocompatibility and can meet requirements for conducting subsequent in vivo experiments on animals.

### Embodiment 8: In Vivo Animal Testing of MHd Material for Promoting Healing of Infected Wounds in Diabetic Mice

In the animal testing, an infected wound model of the diabetic mice is established. All C57 mice (6-week-old, male) are housed in a temperature-controlled animal room for a week after purchase. Streptozotocin (STZ) is dissolved in citric acid buffer (0.1 moles per liter abbreviated as M, potential of Hydrogen abbreviated as pH 4.3) to prepare a 1% STZ solution and stores in the dark. On a day before injection, mice are fasted and treated with water. Mice are intraperitoneally injected with the STZ solution at a dose of 100 milligrams per kilogram (mg/kg) body weight for 5 consecutive days. A week later, blood sugar levels of the mice are detected, and the diabetic mice successfully modeled are selected for the testing. The diabetic mice are anesthetized, depilated, and disinfected, and a full-thickness skin wound with a diameter of 6 mm is created on the back of the diabetic mice. Prepared S. aureus (10⁸ CFU/mL, 20 μL) cultured to a logarithmic phase is dropped into wound sites of the diabetic mice to create an infected wound (recorded as −1 day), and the wounds are attached with 3M (i.e., a brand of Minnesota Mining and Manufacturing Corporation) transparent films and fixed with elastic bandages. Then the diabetic mice are returned to normal feeding. A day later, the diabetic mice are randomly divided into two groups: a control group and an MHd material group. In the MHd material group, the prepared MHd material is attached to the wounds and placed in a high-frequency AMF; infrared imaging is used to observe the temperature of the MHd material in real time, and the MHd material is heated to 50° C. and maintained for 20 minutes with magnetocaloric intervention. In the control group, the wounds are covered with Vaseline gauze and subjected to a treatment of the high-frequency AMF with the same parameters. After interventions, the wounds are attached with 3M transparent films and fixed with elastic bandages. In the MHd material group, on the 3^{rd}, 6^{th}, 9^{th}, and 12^{th} day of the testing, the MHd material is replaced and the interventions are repeated. Before each intervention, photos are taken and recorded to evaluate wound healing status.

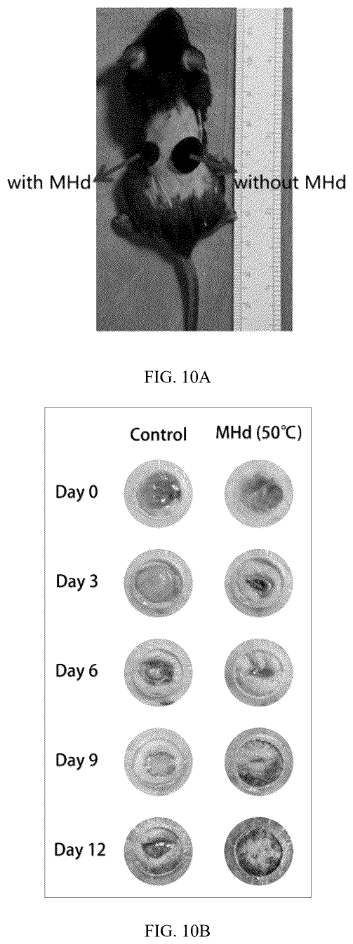

As shown in FIGS. 10A-10B, animal testing results show that the MHd material group has better wound healing status, with complete wound healing on the 12^{th} day and a wound healing rate of 98.4% on the 9^{th} day. In contrast, the wound healing rate of the control group on the 12^{th} day is only 75.5%. On the 3^{rd} day, the wound healing rate of the MHd material group reached 76.7%, which even exceeded the healing rate of the control group on the 12^{th} day. This testing indicates that the MHd material can effectively promote the healing of infected diabetic wounds by providing sustained contraction force for the wounds through magnetocaloric effect and TA synergistic bactericidal property combined with thermoresponsive contraction. In addition, from the overall figure of the wounds, it can be seen that the wound status of the MHd material group is significantly different from the wound status of the control group. Throughout the entire healing cycle, the wounds of the control group are pale yellow and have more exudate, and on the 3^{rd} day, even obvious bacterial biofilm formation can be seen on the wounds of the control group. However, the MHd material group, starting from the 3^{rd} day (two days after the first intervention), shows a much faster healing rate than the control group, and the wound status of the MHd material group changes to a light pink color with less exudate. This testing indicates that a 50° C. intervention in the MHd material group effectively reduces bacterial load that inhibits wound healing. It can be seen that reducing or solving an infection problem is crucial for improving healing effect of infected wounds.

It should be noted that when the claims of the disclosure involve numerical ranges, it should be understood that the two endpoints of each numerical range and any value between the two endpoints can be selected. To avoid repetition, the disclosure describes illustrated embodiments.

Although illustrated embodiments of the disclosure have been described, those skilled in the art may make additional changes and modifications to these embodiments once they have knowledge of basic inventive concepts. Therefore, the attached claims are intended to be interpreted as including illustrated embodiments and all changes and modifications falling within the scope of the disclosure.

What is claimed is:

1. A magnetic hydrogel material with both bactericidal property and thermoresponsive contractility, comprising:
taking a poly(N-isopropylacrylamide) (PNIPAm) copolymerized polyacrylamide (PAm) double-network hydrogel as a matrix, grafting nano clay on the matrix to form a copolymer double-network hydrogel with thermoresponsive contractility, and doping a magnetic substance and tannic acid in the copolymer double-network hydrogel with thermoresponsive contractility to obtain the magnetic hydrogel material; wherein the magnetic substance is triiron tetroxide (Fe₃O₄);
wherein the PNIPAm copolymerized PAm double-network hydrogel is formed through a radical polymerization reaction of N-isopropylacrylamide and acrylamide;
wherein preparation of the magnetic hydrogel material comprises the following steps:
dissolving the N-isopropylacrylamide, a catalyst, the acrylamide, a crosslinking agent, the nano clay, the magnetic substance, and the tannic acid in water to prepare a homogeneous precursor solution; wherein mass concentrations of the N-isopropylacrylamide, the catalyst, the acrylamide, the crosslinking agent, the nano clay, the magnetic substance, and the tannic acid in the homogeneous precursor solution are 8%-12%, 0.2%-0.5%, 1%-3%, 0.1%-0.5%, 3%, 15%, and 1%, respectively; the catalyst is sodium pyrophosphate, and the crosslinking agent is N,N′-methylenebisacrylamide (MBA); and
adding an initiator and an accelerator to the homogeneous precursor solution in a protective gas atmosphere, followed by mixing evenly and polymerizing for 12 hours (h) to 16 h at 4 Celsius degrees (° C.) to 7° C. to obtain the magnetic hydrogel material; wherein the initiator is tetramethylethylenediamine (TMEDA), and the accelerator is an ammonium persulfate solution with a mass concentration in a range of 28% to 32%.

2. The magnetic hydrogel material with both bactericidal property and thermoresponsive contractility as claimed in claim 1, wherein a volume ratio of the water to the initiator to the accelerator is 1000:(1-2.5):(4-6).

3. An application of the magnetic hydrogel material as claimed in claim 1 in preparation of a bactericidal product.

4. The application as claimed in claim 3, wherein the bactericidal property comprises killing Staphylococcus aureus and killing Escherichia coli.

5. An application of the magnetic hydrogel material as claimed in claim 1 in preparation of a dressing for promoting healing of infected diabetic wounds.

6. A dressing for promoting healing of infected diabetic wounds, comprising the magnetic hydrogel material as claimed in claim 1.
